(19) United States
(12) Patent Application Publication
(10) Pub. No.: US 2008/0241290 A1
(43) Pub. Date: Oct. 2, 2008

(54) Sinus relief composition and method of producing the same

(76) Inventor: Wayne Jeffrey Perry, Albany, NY (US)

Correspondence Address: AMIN HALLIHAN, LLC, 444 NORTH ORLEANS, SUITE 400, CHICAGO, IL, 60610, US

(21) Appl. No.: 11/731,656

(22) Filed: Mar. 30, 2007

(51) Int. Cl. A61K 31/195; A61K 36/61; A61P 11/02

(52) U.S. Cl. 424/742; 514/563

(57) ABSTRACT

The present invention provides a sinus relief composition and a method of producing the composition comprising oleoresin capsicum containing capsaicin, dihydrocapsaicin, nordihydrocapsaicin, homodihydrocapsaicin, and homocapsaicin as active ingredients, vegetable glycerin, purified water, eucalyptol, rosemary extract, ascorbic acid, and sea salt for fully relieving and preventing chronic and occasional sinus, allergy, and headache symptoms, including pain, dryness, inflammation, congestion, and coughing.

### FIELD OF THE INVENTION

The present invention relates to a sinus relief composition containing natural capsaicinoids for relieving chronic and occasional sinus, allergy, and headache symptoms, including pain, dryness, inflammation, congestion, and coughing. The present invention is related to a copending application entitled INTRANASAL DRUG DELIVERY SYSTEM filed by the inventor with the United States Patent Office and which disclosure is incorporated herein.

### BACKGROUND OF THE INVENTION

Capsaicinoids are the active components in chili peppers and plants belonging to the Capsicum family. The pungency level of a plant belonging to the Capsicum family is measured on the Scoville scale. The greater the number of Scoville heat units indicates the “hotter” the plant. Capsaicin is a capsaicinoid and the most prevalent capsaicinoid in chili peppers, followed by dihydrocapsaicin, nordihydrocapsaicin, homodihydrocapsaicin, and homocapsaicin. Pure capsaicin ranges between 15,000,000-16,000,000 Scoville units. Dihydrocapsaicin is an irritant and has a similar pungency to capsaicin. Nordihydrocapsaicin, homodihydrocapsaicin, and homocapsaicin are also irritants and have a pungency of about 8,600,000-9,100,000 Scoville units.

Each capsaicinoid and its corresponding chemical structure is shown below.

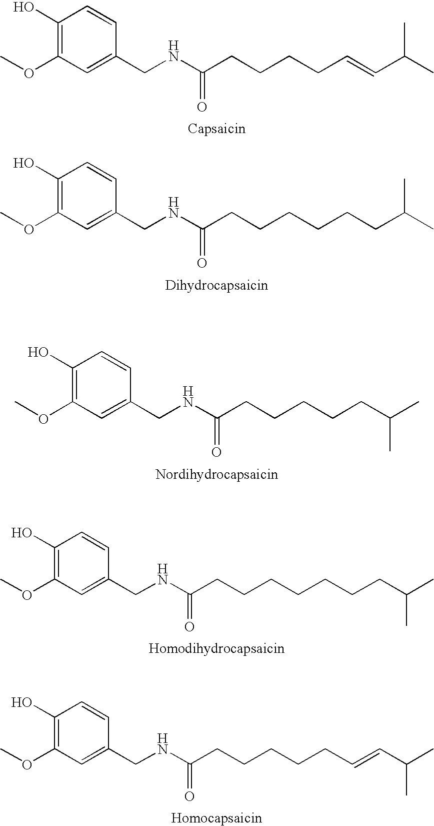

Capsaicinoids are irritants and produce a sensation of burning or hotness when they come in contact with human tissue. Capsaicinoids chemically interact with sensory neurons by binding to thermoreceptor nerve endings inside the body, or receptors in the skin. These receptors can be stimulated with heat or physical abrasion causing chemical signals to pass through the cell membrane and into the cell which causes the neuron to generate its own signal to the brain. The capsaicinoid molecules induce the same effect of producing a burning sensation, but not a chemical burn, that heat or physical abrasion does by binding to the thermoreceptor nerve endings.

Capsaicinoids have many uses such as a heat additive for foods, topical ointments, and non-lethal weapons. Capsaicinoids are used in foods to add spice or “heat”, such as in hot sauce or salsa. Capsaicinoids are also used as a topical ointment to relieve minor aches and pains in muscles and joints, such as symptoms associated with arthritis. Capsaicinoids are also used as an ingredient in the non-lethal weapon commonly known as “pepper spray”, which when sprayed into the eyes or onto the skin is painful to the recipient.

Current treatments for relief of chronic and occasional sinus, allergy and headache symptoms include hydrating, using a steam vaporizer, expectorants, decongestants, cough suppressants, pain medications such as ibuprofen and acetaminophen, antihistamines, saline nasal sprays, air filters, prescription and over the counter drug allergy medicines, steroids and a variety of analgesics. However, these current treatments are mostly unnatural and may require a prescription for utilization.

### SUMMARY OF THE INVENTION

One object of the present invention is for a natural, effective composition for the systemic relief of chronic and occasional sinus, allergy, and headache symptoms.

Another object of the present invention is a composition having components which provide a synergistic effect in the relief of chronic and occasional sinus, allergy and headache symptoms.

Still another object of the present invention is for a composition which provides relief of and prevents chronic and occasional sinus, allergy and headache symptoms.

A further object of the present invention is a composition for the relief of chronic and occasional sinus, allergy, and headache symptoms having the beneficial effects of capsaicinoids without the lingering pain and discomfort of pure pepper.

In accordance with the present invention, a natural composition containing capsaicinoids is used for fully relieving and preventing chronic and occasional sinus, allergy, and headache symptoms, including pain, dryness, inflammation, congestion, and coughing has been developed. In one form, the composition has been developed to be administered intranasally.

The sinus relief composition in accordance with the present invention includes oleoresin capsicum including capsaicin, dihydrocapsaicin, nordihydrocapsaicin, homodihydrocapsaicin, and homocapsaicin as active ingredients has been developed specifically for the purpose of relieving chronic and occasional sinus, allergy and headache symptoms. Additionally, the sinus relief composition relieves symptoms associated with sinus pressure, colds, bacterial infections, sinus congestion, sinus infections, nasal congestion, cluster headaches, congestion headaches and migraines.

In one embodiment of the present invention, the sinus relief composition includes 0.25 mL to 1.2 mL of oleoresin capsicum per 5 gallons of purified water or liquid suspension material.

In another embodiment of the present invention, the sinus relief composition includes oleoresin capsicum including capsaicin, dihydrocapsaicin, nordihydrocapsaicin, homodihydrocapsaicin, and homocapsaicin as active ingredients and having a heat range of 100,000 to 500,000 Scoville units.

In yet another embodiment of the present invention, the sinus relief composition includes oleoresin capsicum including about 67-71% capsaicin, about 20-24% dihydrocapsaicin, about 5-9% nordihydrocapsaicin, about 0.25-2% homodihydrocapsaicin, and about 0.25-2% homocapsaicin.

In still another embodiment of the present invention, the oleoresin capsicum may be water soluble.

Additionally, the sinus relief composition can also include eucalyptol, vegetable glycerin, purified water rosemary extract, ascorbic acid, and sea salt.

The present invention relates also to a method of producing the sinus relief composition. The method includes storing the oleoresin capsicum and purified water between 60 and 65 degrees Fahrenheit; adding the vegetable glycerin to the purified water; agitating the solution to maximize the oleoresin capsicum solubility; adding the ascorbic acid and sea salt and agitating the solution to full solubility; adding the rosemary extract and eucalyptol and agitating the solution to full solubility; and adding the oleoresin capsicum and agitating the solution for at least two minutes and allowing the oleoresin capsicum to reach full solubility without the loss of effectiveness.

These and other embodiments of the present invention are more fully described in connection with the detailed description.

### DETAILED DESCRIPTION OF THE INVENTION

The present invention relates to a natural composition containing capsaicinoids used for fully relieving and preventing chronic and occasional sinus, allergy, and headache symptoms, including pain, dryness, inflammation, congestion, and coughing. The present invention further relates to a composition used to relieve symptoms associated with sinus pressure, colds, bacterial infections, sinus congestion, sinus infections, nasal congestion, cluster headaches, congestion headaches and migraines. The present composition is designed to be administered intranasally.

One embodiment of the present invention relates to a sinus relief composition having oleoresin capsicum including capsaicin, dihydrocapsaicin, nordihydrocapsaicin, homodihydrocapsaicin, and homocapsaicin as active ingredients, combined with eucalyptol, rosemary extract, vegetable glycerin, ascorbic acid, sea salt, and purified water.

The synergistic effect of the components of the sinus relief composition of one form of the invention relieve chronic and occasional sinus, allergy, and headache symptoms without the lingering pain and discomfort of pure pepper. The sinus relief composition of one embodiment of the present invention also relieves symptoms associated with sinus pressure, infections and congestion, colds, bacterial infections, nasal congestion, headaches including cluster, congestion and migraines.

Oleoresin capsicum, a natural resin, is a concentrated extract of capsaicinoids from several Capsicum varieties. The Capsicum variety utilized in the present invention is derived from cayenne pepper plants, which includes Capsicum baccatum and Capsicum frutescens. The oleoresin capsicum includes capsaicin, dihydrocapsaicin, nordihydrocapsaicin, homodihydrocapsaicin, and homocapsaicin as active ingredients and the main heat component of Capsicum variety peppers. Preferably, the oleoresin capsicum utilized in the present invention is all natural, water soluble or soluble in a liquid suspension material. The use of natural oleoresin capsicum including capsaicin, dihydrocapsaicin, nordihydrocapsaicin, homodihydrocapsaicin, and homocapsaicin as opposed to synthetic capsaicin allows for repeated use and effective delivery without too much heat. However, it is within the scope of the present invention that a combination of synthetic capsaicinoids including about 67-71% capsaicin, about 20-24% dihydrocapsaicin, about 5-9% nordihydrocapsaicin, about 0.25-2% homodihydrocapsaicin, and about 0.25-2% homocapsaicin could also be used in the present invention.

The oleoresin capsicum relieves headache and sinus pressure, including cluster, migraine, menstrual, hangover, sinus, and wine headaches. The capsaicinoids in the oleoresin capsicum deplete the chemicals which cause pain and swelling, such as Substance P which is responsible for transmitting pain in the nervous system. The depletion of these chemicals relieves headache pain and the associated sinus pressure.

The oleoresin capsicum relieves sinus and allergy symptoms by flushing out, for example, bacteria, excess mucous, and allergens including dust mites, mold, fungi and pollen. The oleoresin capsicum stimulates the nerve endings in the sinus cavities thereby causing the nerve endings to swell to facilitate release of the toxins, bacteria, excess mucous, and allergens, which relieves the sinus and allergy symptoms. Also, the olfactory nerves contained in the sinus cavities control smell and when these nerves are clogged with bacteria, excess mucous, and allergens caused by a chronic sinus problem, then the nerves fail to regenerate and cause a lost sense of smell. The oleoresin capsicum stimulates the nerve endings, releases the bacteria, excess mucous and allergens, and causes the lost sense of smell to be regained.

The oleoresin capsicum releases capsaicin and other capsaicinoids which naturally suppress the appetite which helps a person from overeating when taken at the moment a person is hungry. Additionally, the oleoresin capsicum increases mental alertness by stimulating nerve fibers and causing the brain to release natural endorphins. This is especially useful for persons experiencing drowsiness while operating a motor vehicle.

The eucalyptol clears nasal congestion, which relieves sinus and allergy symptoms. The eucalyptol helps to clear a pathway for the oleoresin capsicum and allows the oleoresin capsicum to relieve headaches that exist deep within in the sinus cavities. Finally, the eucalyptol is an antiseptic and kills bacteria which are associated with chronic sinus infections.

The rosemary extract is a natural preservative which protects and stabilizes the sinus relief composition. The rosemary extract also opens up the nasal passages and sinus cavities and provides antiseptic properties which kill bacteria in the sinus cavities.

The vegetable glycerin is a natural preservative which protects and stabilizes the sinus relief composition. The vegetable glycerin also assists the capsaicinoids contained in the oleoresin capsicum to maintain their potency and effectiveness for extended periods of time. The vegetable glycerin maintains maximum effectiveness of the sinus relief composition by drawing moisture to the insides of the nasal passages which assists in normal sinus drainage and relieving dryness. Additionally, the vegetable glycerin shortens the length of time of any burning sensation associated with the capsaicinoids in the oleoresin capsicum without reducing its effectiveness. The vegetable glycerin stabilizes the formulation, lengthens the shelf life, does not promote the formation of yeast in the composition, shortens the length of time of the burning sensation associated with the capsaicinoids and preserves the potency of the capsaicinoids for up to five times longer than other natural preservatives, such as aloe vera.

The ascorbic acid naturally preserves the sinus relief composition by adjusting the pH levels. Additionally, the ascorbic acid (vitamin C) supports the immune system and helps to prevent bacteria growth that causes sinus infections.

The sea salt cleans the nasal passages and sinus cavities by flushing out bacteria, and dried or clogged mucous. The sea salt also possesses antiseptic properties.

Finally, the purified water dilutes the sinus relief composition and permits the sinus relief composition to secrete directly into the human body's natural water.

In another embodiment of the present invention, the sinus relief composition may be administered or dispensed through an intranasal spray wherein the sinus relief composition is released in a metered dosage. This administration route instantly depletes pain by permitting the composition to directly enter the nasal passage, blood stream and sinus cavities. Further, the capsaicinoids contained in the sinus relief composition cause the other components to more quickly enter the bloodstream.

In another embodiment of the present invention, the sinus relief composition may be administered through an intranasal spray wherein the intranasal spray is inserted into a first nostril of a human while the second nostril may be held closed by the human. The sinus relief composition may be sprayed one to three times into the first nostril. The sinus relief composition may be sniffed into the nostril. Then the process is repeated for the second nostril. The process may be repeated two to three times per day for both nostrils until the sinus symptoms subside. If the sinus symptoms are severe, then the sinus relief composition may be administered through an intranasal spray, as needed.

In another embodiment of the present invention, the sinus relief composition may be homeopathic wherein the eucalyptol is for congestion and dryness in the throat and the capsaicinoids of the oleoresin capsicum are for sinus pressure, nasal congestion, allergy symptoms, chronic headaches, cold symptoms, bursting headache, coughing, pain, and dryness in the throat. Further, the homeopathic sinus relief composition may include eucalyptol as a tincture.

In accordance with the present invention, the sinus relief composition is comprised of about 0.10 mL to about 1.2 mL oleoresin capsicum per 5 gallons of purified water base or liquid suspension material. The sinus relief composition may include oleoresin capsicum comprised of about 67-71% capsaicin, about 20-24% dihydrocapsaicin, about 5-9% nordihydrocapsaicin, about 025-2% homodihydrocapsaicin, and about 0.25-2% homocapsaicin as active ingredients. Also, the sinus relief composition may include oleoresin capsicum including capsaicin, dihydrocapsaicin, nordihydrocapsaicin, homodihydrocapsaicin, and homocapsaicin as active ingredients and possessing a heat range of 100,000 to 500,000 Scoville units. The oleoresin capsicum including capsaicin, dihydrocapsaicin, nordihydrocapsaicin, homodihydrocapsaicin, and homocapsaicin is present in a non-caustic and safe amount.

In other embodiments of the present invention, the oleoresin capsicum may be water soluble,
the eucalyptol may be Australian,
the rosemary extract may be organic,
the vegetable glycerin may be derived from coconuts and palms,
the sea salt may be a natural solarized salt or a Caribbean fine grain, and
the purified water may be steam distilled to closely match the body's natural water makeup.

The following example of one embodiment of the present invention provides a sinus relief composition having oleoresin capsicum in 5 gallons of purified water or liquid suspension material, and demonstrates the scope of the present invention. The sinus relief composition comprises:

1.2 mL oleoresin capsicum (0.00625% by weight of the total water weight);

15 mL rosemary extract (0.08% by weight of the total water weight);

25 mL eucalyptol (0.13% by weight of the total water weight);

9.6 mL grapefruit seed extract (0.05% by weight of the total water weight);

700 mL vegetable glycerin (3.65% by weight of the total water weight);

100 mL sea salt 0.53% by weight of the total water weight);

160 mL ascorbic acid (0.83% by weight of the total water weight); and

50 mL citric acid (0.26% by weight of the total water weight).

Another example of the present invention relates to a sinus relief composition having oleoresin capsicum in 5 gallons of purified water or liquid suspension material, including:

0.0010% to 0.0063% by weight of the total water weight of oleoresin capsicum;

0.08% by weight of the total water weight of rosemary extract;

0.13% by weight of the total water weight of eucalyptol;

0.05% by weight of the total water weight of grapefruit seed extract;

0.83% by weight of the total water weight of vegetable glycerin;

0.53% by weight of the total water weight of sea salt;

0.83% by weight of the total water weight of ascorbic acid; and

0.26% by weight of the total water weight of citric acid.

In another embodiment, the capsaicinoids in the oleoresin capsicum may have a low heat level wherein the potency of the capsaicinoids is not as strong due to a lower dosage of the oleoresin capsicum. This embodiment is suited for daily use and for preventing and relieving mild sinus and allergy symptoms, such as dull headaches and minor congestion and pressure.

In an example of a low heat level embodiment, the sinus relief composition includes oleoresin capsicum in 5 gallons of purified water and comprises:

0.0010% to 0.0013% by weight of the total water weight of oleoresin capsicum;

0.08% by weight of the total water weight of rosemary extract;

0.13% by weight of the total water weight of eucalyptol;

0.05% by weight of the total water weight of grapefruit seed extract;

3.65% by weight of the total water weight of vegetable glycerin;

0.53% by weight of the total water weight of sea salt;

0.83% by weight of the total water weight of ascorbic acid; and

0.26% by weight of the total water weight of citric acid.

In an example of low heat level embodiment, the sinus relief composition includes oleoresin capsicum in 5 gallons of purified water and comprises:

0.25 mL oleoresin capsicum (0.001% by weight of the total water weight);

15 mL rosemary extract (0.08% by weight of the total water weight);

25 mL eucalyptol (0.13% by weight of the total water weight);

9.6 mL grapefruit seed extract (0.05% by weight of the total water weight);

700 mL vegetable glycerin (3.65% by weight of the total water weight);

100 mL sea salt 0.53% by weight of the total water weight);

160 mL ascorbic acid (0.83% by weight of the total water weight); and

50 mL citric acid (0.26% by weight of the total water weight).

One embodiment of the present invention is a sinus relief kit having a sinus relief composition having oleoresin capsicum including capsaicin, dihydrocapsaicin, nordihydrocapsaicin, homodihydrocapsaicin, and homocapsaicin as active ingredients, vegetable glycerin, purified water, eucalyptol, rosemary extract, ascorbic acid, and sea salt; and an instruction for a human to administer the sinus relief composition intranasally. One form of the sinus relief kit includes the sinus relief composition described above and an instruction for a human to administer the sinus relief composition intranasally. In another embodiment, administering the sinus relief composition may also include inserting an intranasal spray into a first nostril of a human; holding a second nostril closed; spraying the sinus relief composition one to three times into the first nostril; sniffing the sinus relief composition into the nostril; repeating for the second nostril; and repeating two to three times per day for first and second nostrils until sinus relief is provided.

The present invention also includes a method of producing the sinus relief composition. One embodiment of the method of producing the sinus relief composition includes storing the oleoresin capsicum and purified water between 60 and 65 degrees Fahrenheit, which increases the water solubility of the oleoresin capsicum. The method further includes adding the vegetable glycerin to the purified water first; agitating the first solution to maximize the oleoresin capsicum solubility; adding the ascorbic acid and sea salt to the first solution to make a second solution and agitating the second solution to maximum solubility; adding the rosemary extract and eucalyptol to the second solution to make a third solution and agitating the third solution to maximum solubility; and adding the oleoresin capsicum to the third solution and making a fourth solution and agitating the fourth solution and allowing the oleoresin capsicum to reach maximum solubility without losing effectiveness.

In still another embodiment of the present invention, agitating the first solution to maximize the oleoresin capsicum solubility may include agitating the first solution two times wherein each agitation lasts one minute. In another embodiment, agitating the fourth solution permits the oleoresin capsicum to reach maximum solubility without losing effectiveness may include agitating the fourth solution for at least two minutes.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

We claim:

1. A sinus relief composition for administering to a human by secreting directly into the human's natural water system, comprising:
a liquid suspension material comprising purified water, and
oleoresin capsicum including capsaicin, dihydrocapsaicin, nordihydrocapsaicin, homodihydrocapsaicin, and homocapsaicin as active ingredients.

2. The composition in accordance with claim 1, further comprising:
vegetable glycerin;
eucalyptol;
rosemary extract;
ascorbic acid; and
sea salt.

3. The composition in accordance with claim 1, wherein the composition is administered intranasally.

4. The composition in accordance with claim 1, wherein the oleoresin capsicum is in a concentration of between about 0.10 mL to about 1.2 mL per 5 gallons of the purified water.

5. The composition in accordance with claim 1, wherein the oleoresin capsicum has a heat level of between about 100,000 to 500,000 Scoville units.

6. The composition in accordance with claim 1, wherein the purified water is steamed distilled to match the human's natural water molecule to facilitate the water solubility of the oleoresin capsicum.

7. The composition in accordance with claim 1, wherein the oleoresin capsicum is comprised of 67-71% capsaicin, about 20-24% dihydrocapsaicin, about 5-9% nordihydrocapsaicin, about 0.25-2%% homodihydrocapsaicin, and about 0.25-2% homocapsaicin.

8. The composition in accordance with claim 2, wherein the eucalyptol is a tincture.

9. The composition in accordance with claim 2, wherein the eucalyptol is Australian.

10. The composition in accordance with claim 2, wherein the rosemary extract is organic.

11. The composition in accordance with claim 2, wherein the vegetable glycerin is derived from coconuts and palms.

12. The composition in accordance with claim 2, wherein the sea salt is naturally solarized.

13. A sinus relief composition containing purified water obtained by steam distillation, comprising:
0.0010% to 0.0063% by weight of the total water weight of oleoresin capsicum;
0.08% by weight of the total water weight of rosemary extract;
0.13% by weight of the total water weight of eucalyptol;
0.05% by weight of the total water weight of grapefruit seed extract;
3.65% by weight of the total water weight of vegetable glycerin;
0.53% by weight of the total water weight of sea salt;
0.83% by weight of the total water weight of ascorbic acid; and
0.26% by weight of the total water weight of citric acid.

14. The composition of claim 13 wherein the oleoresin capsicum is between 0.0010% to 0.0013% by weight of the total water weight.

15. A method of producing a sinus relief composition comprising the steps of:
adding vegetable glycerin to purified water to make a first solution;
agitating the first solution;
adding ascorbic acid, sea salt, rosemary extract, and eucalyptol to the first solution to make a second solution;
agitating the second solution;
adding oleoresin capsicum to the third solution to make a fourth solution; and
agitating the fourth solution.

16. The method in accordance with claim 15, wherein the oleoresin capsicum and purified water are stored between 60 to 65 degrees Fahrenheit.

17. The method in accordance with claim 15, wherein agitating the first solution is done two times for one minute each time.

18. The method in accordance with claim 15, wherein agitating the fourth solution is done for at least two minutes.

19. A method of providing sinus relief comprising:
administering intranasally a sinus relief composition comprising oleoresin capsicum including capsaicin, dihydrocapsaicin, nordihydrocapsaicin, homodihydrocapsaicin, and homocapsaicin as active ingredients.

20. The method in accordance with claim 19, wherein administering intranasally further comprises:
inserting an intranasal spray into a first nostril of a human;
holding a second nostril closed;
spraying the sinus relief composition into the first nostril;
inserting the intranasal spray into the second nostril of the human;
holding the first nostril closed; and
spraying the sinus relief composition into the second nostril.

21. A sinus relief kit comprising:
a sinus relief composition comprising oleoresin capsicum including capsaicin, dihydrocapsaicin, nordihydrocapsaicin, homodihydrocapsaicin, and homocapsaicin as active ingredients; and
an instruction for a human to administer the sinus relief composition intranasally by said sinus relief composition into said first and said second nostril between one to two times to secrete the composition directly into the human's natural water system.

22. The sinus relief kit in accordance with claim 21, wherein administering the sinus relief composition intranasally further comprises:
inserting an intranasal spray into a first nostril of a human;
holding a second nostril closed;
spraying the sinus relief composition one to three times into first nostril;
inserting the intranasal spray into the second nostril of the human;
holding the first nostril closed; and
spraying the sinus relief composition one to three times into second nostril.
